(19) United States
(12) Patent Application Publication
(10) Pub. No.: US 2005/0203192 A1
(43) Pub. Date: Sep. 15, 2005

(54) Process and composition for treating viral infections

(76) Inventor: Laran Lerner, Northville, MI (US)

Correspondence Address: DICKINSON WRIGHT PLLC, 1901 L. STREET NW, SUITE 800, WASHINGTON, DC, 20036, US

(21) Appl. No.: 11/078,222

(22) Filed: Mar. 11, 2005

Related U.S. Application Data
(60) Provisional application No. 60/552,585, filed on Mar. 11, 2004.

(51) Int. Cl. A61K 31/03; A61K 31/02

(52) U.S. Cl. 514/743; 514/749

(57) ABSTRACT

A method and composition for treating herpes group viral infection in a host that reduces the duration and severity of lesions associated therewith. A therapeutically effective amount of a halogenated hydrocarbon compound is applied topically to the affected tissue in a dosage or frequency sufficient to speed healing of the affected tissue. Halogenated hydrocarbons may include organochlorides (such as methyl chlorides and ethyl chlorides), alkyl chlorides, alkyl bromides, and alkyl fluorides (such as methyl fluoride). Other compounds known to be effective in decreasing the recurrence of the herpes group virus in a dosage to speed healing of infected tissue may also be combined and applied. Such other compounds may include acyclovir, famcyclovir, viral thymidine kinase inhibitor, and other partially effective herpes simplex viral recurrence inhibitors known in the art.

This application claims priority from Provisional Application No. 60/552,585, filed on Mar. 11, 2004, the entire contents of which are herein incorporated by reference to the extent allowed by law.

### FIELD OF THE INVENTION

The present invention relates generally to a process and composition for treating viral infections in a host such as A mammal, and more particularly to a process for treating herpes group viral infections. Most particularly, the present invention relates to topical and intradermal application of chlorinated hydrocarbons to treat active herpes simplex lesions in humans.

### BACKGROUND OF INVENTION

Infections relating to Herpes group viruses are well known in clinical medicine. Herpes viruses are DNA viruses known to infect a wide variety of organisms. One particular feature of herpes virus biology relates to the virus' tendency to remain in ganglia of an infected host in a latent state after an initial infection. Various trigger factors can induce reactivation of the viral infection, and outbreaks of active lesions can occur throughout the life of a once infected individual. This latency period is understood to allow the virus to escape total destruction by the host immune system, and may contribute to resistance to various drug therapies.

There are four herpes group viruses known to infect and cause disease in humans. These are (1) herpes simplex virus types I and II; (2) varicella-zoster virus; (3) cytomegalovirus; and (4) Epstein-Barr virus. Clinical conditions most commonly associated with herpes simplex type I are the familiar cold sores and fever blisters appearing around the mouth and other mucous membranes of infected individuals, known clinically as herpes labialis. Herpes simplex type II is more commonly associated with lesions of the genital areas, and infections are known clinically as herpes progenitalis. Varicella-zoster is associated with the familiar diseases of chicken-pox and shingles in humans.

The herpes simplex virus (HSV) is a large (150-200 nm) DNA virus which consists of approximately 152,000 base pairs of double stranded DNA encapsulated in a proteinaceous capsid. The capsid is surrounded by a less well defined structure known as the tegument. The virus is contained in a host cell derived lipid bilayer, which is studded with virus specific glycoproteins and integral membrane proteins.

Recurrent herpes genitalis is estimated to affect over 45 million people in the USA alone. (Fleming et al NEJM October 97). The incidence is increasing, leading to a massive reservoir for transmission of infection to sexual partners and new-born infants. Once infected, over 95% of patients who have proven primary herpes genitalis have had at least one recurrence with an average of 5-6 recurrences per year. Approximately 25% of people will have recurrences at monthly intervals accompanied by extremely troublesome physical discomfort and (often) psychological upset. Current therapy for genital herpes revolves around psychological support, education, barrier methods of contraception and treatment on a regular basis with the expensive anti-viral drugs such as Aciclovir, Penciclovir and Famciclovir. These are not tolerated by all sufferers and in some cases are ineffective.

Herpes simplex infection may be spread from mother to fetus. This usually occurs during parturition but can rarely occur in-utero. The rate of transmission depends on whether the mother is suffering primary herpes, where the transmission rate is 50% or recurrent herpes where the transmission rate is 8%. If any lesions are visible during the onset of labour, this would be an indication for undertaking a caesarean section.

About 50-60% of infants with neonatal Herpes simplex virus infection are born to mothers with no history of genital herpes infection. Neonatal herpes simplex virus infection is very serious. Over 70% of cases present infections localised to the eyes, skin or mouth within 3-30 days of birth. Unfortunately, over three quarters of those infected will progress to disseminated or CNS involvement which carries a very high morbidity and mortality rate. The herpes viruses have a role in other pathology, such as acute encephalitis, ulcerative colitis, and Bells Palsy and cancer.

As early as 1975 a link between herpes simplex and senile dementia was suggested. (Libikova Acta Virologica 19(6): 493-5, 1975). In this study, 47% of patients suffering with senile dementia had neutralizing antibodies to HSV 1 in their cerebrospinal fluid, compared to none of mentally retarded adolescents. Serum levels of antibodies were elevated in 61% of demented patients compared with 31% of normal controls. More recent work (Jamieson et al Journal of Pathology 167(4): 365-Aug. 8, 1991) used PCR (polymerase chain reaction) to detect thymidine kinase from viral DNA. There was a significant anatomically specific presence of herpes simplex in the brains of those with senile dementia with virus being commonly detected in the hippocampus and the cortex but universally absent in the occipital cortex.

Herpes group virus is not only extremely widespread within the general population but its pathological effects can be just as diverse highlighting the urgent need for effective treatments.

Myriad formulations and processes for treating herpes group infections have been developed over the years. Orally administered and topical preparations are known, however, most have achieved at best only marginal success. In general, viral infections have historically proven difficult to treat, and herpes simplex infections especially so. This is believed to result, at least in part, from the virus' insulation from a host's immune system during the latency period inside the host's ganglia.

### SUMMARY OF THE INVENTION

The present invention to provide a method and composition for treating herpes group viral infection in a host that reduces the duration and severity of lesions associated therewith.

Accordingly, the present invention provides a method and composition for treating herpes group infections in a host comprising the step of applying a therapeutically effective amount of a halogenated hydrocarbon compound to the affected tissue in a dosage or frequency sufficient to speed healing of the affected tissue. Halogenated hydrocarbons may include organochlorides (such as methyl chlorides and ethyl chlorides), alkyl chlorides, alkyl bromides, and alkyl fluorides (such as methyl fluoride).

Additionally, the invention also provides a method for treating herpes group viral infections in a host wherein the application of the therapeutically effective amount of a halogenated hydrocarbon compound is combined with other compounds known to be effective in decreasing the recurrence of the herpes group virus in a dosage to speed healing of infected tissue. Such other compounds may include acyclovir, famcyclovir, viral thymidine kinase inhibitor, and other partially effective herpes simplex viral recurrence inhibitors known in the art.

One aspect of the invention is to configure the composition to evaporate relatively rapidly when applied topically.

Another aspect of the present invention provides a method for treating a herpes group virus in a host. This method includes administering to a host a therapeutically effective amount of a composition against said herpes group virus having at least one halogenated hydrocarbon compound.

Other features and advantages of the present invention will become more apparent to persons having ordinary skill in the art to which the present invention pertains from the following description.

### DETAILED DESCRIPTION OF THE INVENTION

The present invention provides a method for treating viral infections, in particular using halogenated hydrocarbon compounds such as alkyl or organo-halides, preferably organochlorides. Alkyl halides are presently known and used, for example, as vapocoolant materials for topical pain treatment. Exemplary compounds include ethyl chloride (chloroethane) and fluorimethane (methyl fluoride). Less preferred compounds include more complex alkyl halides such as chloro-di-fluoro-methane. Chloro-ethane and spays sold under the tradename FLUORI-METHANE (dichlorodifluoromethane 15% and trichloromonofluoromethane 85%) are available commercially from the Gebauer Company of Cleveland, Ohio. Other vapocoolant sprays are commercially available, such as those sold under the tradename FRIGIDERM from Delasco Corp. of Council Bluffs, Iowa. Representatives from this class of compounds are known to evaporate relatively rapidly when applied externally, causing cooling and contracting of blood vessels in the skin and locally alleviating pain. It was heretofore unknown, however, that the administration of vapocoolant sprays could speed the healing, and prevent recurrence of herpes related lesions.

The class of suitable compounds is relatively broad, and includes in addition to alkyl chlorides, alkyl bromides, and alkyl fluorides. Particularly preferred alkyl chlorides are methyl and ethyl chlorides. Certain alkyl halides are known to be toxic when ingested or absorbed through the skin. Where the relatively more toxic alkyl halides are used, they may be diluted in a liquid carrier to a concentration that is harmless as defined in the literature, for example, Hawley's Condensed Chemical Dictionary, Twelfth Edition. Preservatives and other additives may also be present in the therapeutic composition such as, for example, carriers, other analgesics, antimicrobials, anti-oxidants, chelating agents, inert gases, and the like.

The term “therapeutically effective amount” as used herein refers to an amount of the alkyl halide sufficient to decrease the healing time for active herpes lesions and/or to decrease the frequency of reactivation of a herpes virus infection. The term “therapeutically effective amount” therefore includes, for example, an amount of an alkyl halide sufficient to reduce the healing time or recurrence of lesions, and preferably to reduce by at least 50% the healing time or frequency of lesion recurrence. The dosage ranges for the administration of the alkyl halide are those that produce the desired effect. A person of ordinary skill in the art, given the teachings of the present specification, may readily determine suitable dosage ranges, by varying concentration of alkyl halide in the mixture applied and/or by varying the frequency of application. The dosage can be adjusted by the individual physician in the event of any contraindications. In any event, the effectiveness of treatment can be determined by monitoring the recurrence of lesions by methods well known to those in the field. The alkyl halide can also be combined with other compounds known to be effective in decreasing the recurrence of herpes viruses, e.g., acyclovir, famcyclovir, viral thymidine kinase inhibitor, and other partially effective HSV recurrence inhibitors.

Broadly, the presently described process is a new use for the described class of known compounds, the process comprising the step of applying an alkyl halide such as ethyl chloride in a therapeutically effective amount to herpes lesions to improve healing of the lesions as compared to healing without the treatment. The method is applicable to mammals, most particularly humans, and is contemplated for topical application to lesions resulting from infection by any known herpes virus, in particular (1) herpes simplex virus types I and II; (2) varicella-zoster virus; (3) cytomegalovirus; and (4) Epstein-Barr virus. The present process is contemplated for use in treating herpes infections affecting the skin and mucous membranes, including the eyes, mouth, intestinal, nasal, anal, vaginal and urethral membranes.

In a preferred embodiment, the alkyl halide is applied in a spray form to active herpes lesions one or more times per day, at a frequency and in a concentration sufficient to speed healing. As an alternative to spraying the alkyl halide, it may be applied, for example, with a spray-saturated cotton ball or swab. The alkyl halide may also be administered to a patient by any other suitable means, including subcutaneous and topical administration. The alkyl halide may also be administered transdermally, for example, in the form of a slow-release subcutaneous implant or an epidermal patch.

As used herein, the term “active” should be understood to mean lesions wherein the patient's skin is broken. This particular application to broken tissue is not limiting, however, the composition might instead be applied, for example prophylactically, to tissue areas prone to herpes outbreaks or currently experiencing a presymptomatic outbreak. Prophylactic treatments might be made when the patient has experienced various “trigger factors,” such as immune stress due to chemotherapeutic cancer or tumor treatments, or immunosuppressive drugs used to prevent organ rejection in transplants. Similarly, prophylactic treatment may be undertaken when the patient experiences other potential trigger factors such as chapping of the lips. During a treatment program, it may also be desirable to continue application of the composition to the affected area after the herpes lesions heal over.

Alternative embodiments are contemplated, as described above wherein the alkyl halide is delivered intradermally rather than topically. The compound may be delivered by hypodermic injection with a suitable carrier fluid into and around the affected tissue, or the compound may be delivered by some other means such as by agitation of the affected tissue and swabbing the area with an impregnated delivery device.

The present description is for illustrative purposes only, and should not be construed to limit the scope of the present invention in any way. Thus, those skilled in the art will appreciate that various departures might be made from the presently disclosed embodiments without departing from the full and fair scope of the present invention.

1. A therapeutic composition comprising at least one halogenated hydrocarbon compound in a dosage sufficient to speed healing of tissue affected with a herpes group virus.

2. The therapeutic composition of claim 1, further comprising a second compound known to be effective in decreasing the recurrence of said herpes group virus.

3. The therapeutic composition of claim 1, wherein said at least one halogenated hydrocarbon compound is a composition known to evaporate relatively rapidly when applied topically.

4. The therapeutic composition of claim 3, wherein said at least one halogenated hydrocarbon compound is an organochloride.

5. The therapeutic composition of claim 4, wherein said organochloride is selected from the group consisting of methyl and ethyl chlorides.

6. The therapeutic composition of claim 3, wherein said at least one halogenated hydrocarbon compound is selected from the group consisting of an alkyl chlorides, alkyl bromides, and alkyl fluorides.

7. The therapeutic composition of claim 6, wherein said alkyl fluoride is methyl fluoride.

8. The therapeutic composition of claim 2, wherein said second compound known to be effective in decreasing the recurrence of said herpes group virus is selected from the group consisting of acyclovir, famcyclovir, viral thymidine kinase inhibitor, and other partially effective herpes simplex viral recurrence inhibitors.

9. The therapeutic composition of claim 2, wherein said at least one halogenated hydrocarbon compound is a composition known to evaporate relatively rapidly when applied externally.

10. The therapeutic composition of claim 9, wherein said at least one halogenated hydrocarbon compound is an organochloride.

11. The therapeutic composition of claim 10, wherein said organochloride is selected from the group consisting of methyl and ethyl chlorides.

12. The therapeutic composition of claim 9, wherein said at least one halogenated hydrocarbon compound is selected from the group consisting of alkyl chlorides, alkyl bromides, and alkyl fluorides.

13. The therapeutic composition of claim 12, wherein said alkyl fluoride is methyl fluoride.

14. A method for treating a herpes group virus in a host, said method comprising the step of administering to said host a therapeutically effective amount of a composition against said herpes group virus having at least one halogenated hydrocarbon compound.

15. The method of claim 14, wherein said composition further comprises a second compound known to be effective in decreasing the recurrence of said herpes group virus.

16. The method of claim 15, wherein the herpes group virus is selected from the group consisting of herpes simplex virus types I and II, varicella-zoster virus, cytomegalovirus, and Epstein-Barr virus.

17. The method of claim 14, wherein said at least one halogenated hydrocarbon compound is a composition known to evaporate relatively rapidly when applied externally.

18. The method of claim 17, wherein said at least one halogenated hydrocarbon compound is an organochloride.

19. The method of claim 18, wherein said organochloride is selected from the group consisting of methyl chlorides and ethyl chlorides.

20. The method of claim 14, wherein said at least one halogenated hydrocarbon compound is selected from the group consisting of an alkyl chlorides, alkyl bromides, and alkyl fluorides.

21. The therapeutic composition of claim 20, wherein said alkyl fluoride is methyl fluoride.

22. The method of claim 15, wherein said at least one halogenated hydrocarbon compound is a composition known to evaporate relatively rapidly when applied externally.

23. The method of claim 22, wherein said at least one halogenated hydrocarbon compound is an organochloride.

24. The method of claim 23, wherein said organochloride is selected from the group consisting of methyl and ethyl chlorides.

25. The method of claim 22, wherein said at least one halogenated hydrocarbon compound is selected from the group consisting of an alkyl chlorides, alkyl bromides, and alkyl fluorides.

26. The therapeutic composition of claim 25, wherein said alkyl fluoride is methyl fluoride.

27. The method of either claim 14 or 15, wherein said treating is selected from the group consisting of topical and intradermal applications.
